(19) United States
(12) Patent Application Publication
(10) Pub. No.: US 2025/0115549 A1
(43) Pub. Date: Apr. 10, 2025

(54) FLOW SYNTHESIS PROCESS FOR THE PRODUCTION OF SULFONYLUREA COMPOUNDS

(71) Applicant: NELSON MANDELA UNIVERSITY, Port Elizabeth (ZA)

(72) Inventors: Paul WATTS, Port Elizabeth (ZA); Cloudius Ray SAGANDIRA, Port Elizabeth (ZA)

(21) Appl. No.: 18/683,275

(22) PCT Filed: Aug. 12, 2022
(86) PCT No.: PCT/IB2022/057538

§ 371 Date: Feb. 13, 2024

(30) Foreign Application Priority Data
Aug. 13, 2021 (GB) 2111677.7

(51) Int. Cl. C07C 315/04

(52) U.S. Cl. CPC C07C 315/04

(57) ABSTRACT

This invention provides a flow synthesis process for producing sulfonylurea compounds of formula (1), including gliclazid, chlorpropamide and tolbutamide, and pharmaceutically acceptable salts thereof.

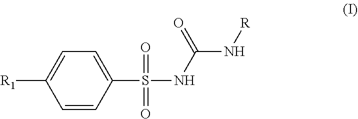

### INTRODUCTION

This invention relates to a process for producing sulfonylurea compounds, including gliclazide, chlorpropamide and tolbutamide, and pharmaceutically acceptable salts thereof, in particular to a flow synthesis process for producing these compounds.

### BACKGROUND

Sulfonylureas, discovered by Janbon and co-workers in 1942, have been extensively used for treatment of type 2 diabetes for nearly 50 years. Despite several anti-diabetic agents on the market, sulfonylureas remain one of the most prescribed due to affordability, possibility of mono-dosing and presence of an association with metformin in the same tablet.

Diabetes mellitus is an endocrinological disorder that causes high blood sugar. According to the International Diabetes Federation (IDF) 425 million people had diabetes in 2017 worldwide with 1.8 million cases being reported in South Africa. Furthermore, it is predicted that about 552 million people worldwide will have diabetes by 2030.

Gliclazide 1a, chlorpropamide 1b and tolbutamide 1c are some of the sulfonylurea drugs used for the treatment of diabetes mellitus. Although there are various synthetic approaches in literature towards these drugs, these processes are all inefficient.

Further, existing synthesis methodologies for the production of these compounds have essentially been based on standard stirred batch reactor type processes, wherein significant volumes of organic solvents are used.

Micro reactor technology (MRT), more recently branded ‘flow chemistry’, is an emerging technique that enables those working in research and development to rapidly screen reactions utilising continuous flow, leading to the identification of reaction conditions that are suitable for use at a production level. Furthermore, in addition to using conventional reaction methodology, the inherent safety associated with the use of small reactor volumes enables users to employ reaction conditions previously thought to be too hazardous for use within a production environment; such as extreme reaction conditions or the use/generation of ‘hazardous’ compounds. Consequently, the type of reactions available to the chemist increases through the use of this technology.

Although it is known to be desirable to transfer batch processes to flow synthesis, it often requires significant reaction condition and reagent modifications which are not necessarily obvious, considering the vast amount of permutations in reagents and reaction conditions and the unpredictable nature of chemistry in general. Furthermore, flow synthesis methodology has inherent challenges that has to be overcome, in particular for continuous multistep reactions.

The present invention seeks to address some of the shortcomings of the prior art by providing new flow chemistry processes for producing sulfonylurea compounds, including gliclazide, chlorpropamide and tolbutamide.

### SUMMARY OF THE INVENTION

According to a first aspect to the present invention there is provided a flow synthesis process for producing a sulfonylurea compound of the Formula 1 or its pharmaceutically acceptable salts,

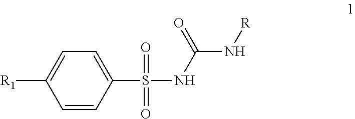

the process comprising the steps of:

a) preparing the carbamate of Formula 2

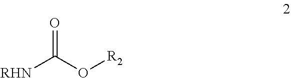

by reacting free amine RNH₂ or amine HCl salt with haloformate of Formula 4 in the presence of a base

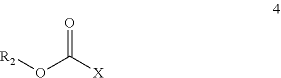

b) reacting the carbamate of Formula 2 with the sulfonamide of Formula 3 in the presence of a base

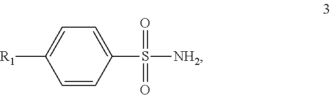

wherein

R₂ is selected from aryl and alkyl,

R₁ is selected from Cl and —CH₃,

R is selected from propyl, butyl, and

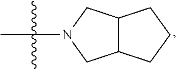 [SMILES: *N1CC2CCCC2C1]

and

X is selected from Cl, Br, and I.

In one embodiment, X is Cl.

In one embodiment, the process is a continuous multi-step process without the isolation of any intermediates.

In a preferred embodiment, in step (a) the free amine RNH₂ or amine HCl salt to base molar ratio is about 1:1.5 to about 1:3.

Preferably, the reaction of step (a) is performed using aryl chloroformate.

More preferably, the reaction of step (a) is performed using phenyl chloroformate.

In one embodiment, the reaction of step (a) is performed using aryl iodoformate or aryl bromoformate, in particular phenyl iodoformate phenyl bromoformate.

Preferably, the reaction of step (a) is performed in an organic solvent selected from the group consisting of chloroform, dichloromethane, acetonitrile, and mixtures thereof.

More preferably, the reaction of step (a) is performed in chloroform.

In one embodiment, the base in step (a) and step (b) is independently selected from 1,8-Diazabicyclo[5.4.0]undec-7-ene (DBU), trimethylamine (TEA), and tributyl amine (TBA).

In one embodiment, the base used in step (a) and step (b) is the same.

Preferably, the reaction of step (a) is thermally controlled to a temperature equal to about room temperature or less.

More preferably, the reaction of step (a) is thermally controlled to a temperature between about 0° C. and about room temperature.

In a preferred embodiment, the free amine RNH₂ or amine HCl salt has a concentration of about 0.1M to about 2M.

In a preferred embodiment, the sulfonamide has a concentration of about 0.1M to about 2M.

In a preferred embodiment, in step (b) the sulfonamide to carbamate molar ratio is about 1:1 to about 1:2.

In a particularly preferred embodiment, in step (b) the sulfonamide to carbamate molar ratio is about 1:1.5.

In a preferred embodiment, in step (b) the sulfonamide to base molar ratio is about 1:2 to about 1:5.

Preferably, the reaction of step (b) is thermally controlled to a temperature between about 70° C. and about 100° C.

More preferably, the reaction of step (b) is thermally controlled to a temperature of about 80° C.

Preferably, the reaction of step (b) is performed in an organic solvent selected from the group consisting of chloroform, dichloromethane, acetonitrile, and mixtures thereof.

More preferably, the reaction of step (b) is performed in acetonitrile.

### BRIEF DESCRIPTION OF THE DRAWINGS

The invention will now be described in more detail with reference to the following non-limiting embodiments and figures in which:

FIG. 1 shows the effect of the molar equivalent ratio of base (DBU) and carbamate 2a to sulfonamide 3a in the reaction (step (b)) towards gliclazide 1a;

FIG. 2 shows the effect of variations in temperature, molar equivalents, and residence time of the reaction of step (b) towards gliclazide 1a; and

FIG. 3 shows the effect of variations in molar equivalents and residence time of the reaction of step (b) towards chlorpropamide 1b and tolbutamide 1c.

### DETAILED DESCRIPTION OF A PREFERRED EMBODIMENT

The present invention will now be described more fully hereinafter with reference to the accompanying figures, in which some of the non-limiting embodiments of the invention are shown.

The invention as described hereinafter should not be construed to be limited to the specific embodiments disclosed, with slight modifications and other embodiments intended to be included within the scope of the invention.

Although specific terms are employed herein, they are used in a generic and descriptive sense only and not for purposes of limitation.

As used herein, throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having”, “including”, and variations thereof used herein, are meant to encompass the items listed thereafter, and equivalents thereof as well as additional items.

The present invention provides for a flow synthesis process for producing sulfonylurea compounds and pharmaceutically acceptable salts thereof, in particular to a flow synthesis process for producing gliclazide, chlorpropamide and tolbutamide. In a particularly preferred embodiment, the invention provides for a continuous multistep flow synthesis process for producing gliclazide, chlorpropamide and tolbutamide without the isolation of any intermediates.

Although it is known to be desirable to transfer exiting batch processes or methodologies to flow synthesis systems, it requires significant reaction condition and reagent type modifications which are not obvious to the skilled person, in particular considering the endless permutations in reagents and reaction conditions available. Furthermore, flow synthesis has inherent challenges that has to be overcome, in particular for continuous multistep reactions. The inventors of the present invention overcame these challenges, and developed highly efficient and rapid methods for the synthesis of various sulfonylurea compounds, as disclosed herein.

As shown in Scheme 1 below, the inventors have envisioned the preparation of sulfonylurea 1 (Gliclazide 1a, chlorpropamide 1b and tolbutamide 1c) from the reaction of the key carbamate 2 intermediate with sulfonamide 3. Whilst sulfonamide 3 is commercially available, carbamate 2 is not readily available. Carbamate 2 was prepared from the treatment of free amine 5 or amine HCl salt with chloroformate 4.

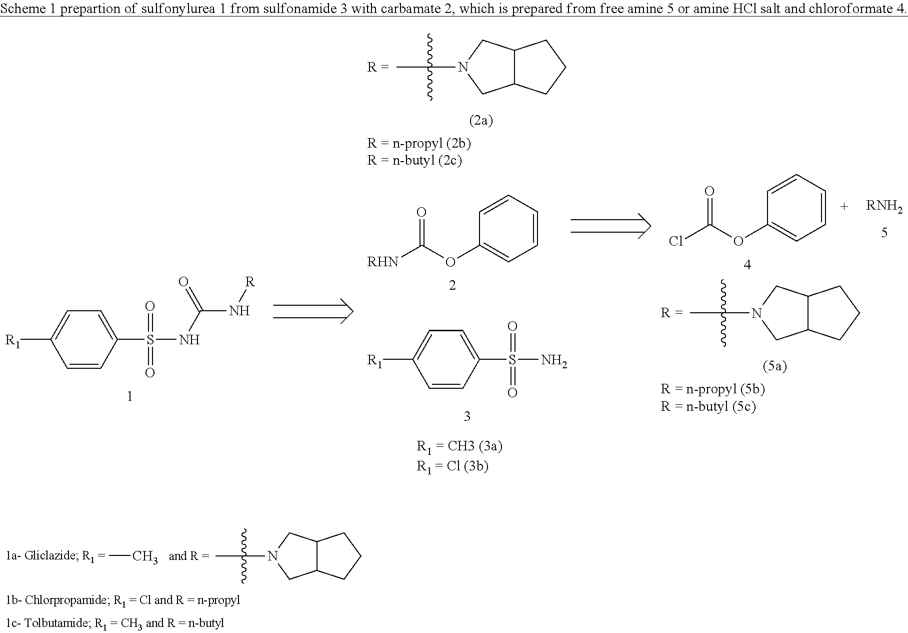

### General Experimental Procedures

Chemicals were supplied by Sigma Aldrich, Merck and Industrial Analytical and were used as received. Anhydrous solvents were supplied by Sigma Aldrich, and maintained by drying over appropriately activated molecular sieves during use.

Column chromatography was performed using Fluka Chemie silica gel 60 as the stationary phase, and mixtures of ethyl acetate and hexane of varying polarity were used as the mobile phase. Unless otherwise stated, thin layer chromatography (TLC) was done using Merck Kieselgel 60 HF254 aluminium backed TLC plates with mixtures of ethyl acetate and hexane of varying polarity as eluent. TLC visualisation was done by fluorescence on exposure to short wave ultra violet (UV) light (λ254 nm) in a Camag UV cabinet.

Nuclear magnetic resonance (NMR) spectra were recorded at room temperature as solutions in deuterated chloroform (CDCl₃) or deuterated dimethyl sulfoxide (DMSO-d₆). A Bruker Avance-400 spectrometer (400 MHz) was used to record the spectra and the chemical shifts are reported in parts per million (ppm) with coupling constants in Hertz (Hz).

Infra-red spectra were recorded from 4000 to 500 cm⁻¹ using a Bruker spectrometer and peaks (Vₘₐₓ) reported in wavenumbers (cm⁻¹). Melting points of all compounds were determined using a Staurt Melting Point Apparatus SMP30.

High performance liquid chromatography (HPLC) data was obtained using Agilent 1220 with a UV/Vis detector: HPLC analysis was performed on ACE Generix 5 C18(2) column (150 mm×4.6 mm i.d.) at ambient temperature using an isocratic system. The mobile phase consisted of 70% acetonitrile and 30% water. The sample injection volume was 1 μl, eluted at a flow rate of 1.5 ml/min and detected at 230 nm with a run time of 7 min.

The collected carbamate solutions were washed with water and extracted into DCM. The products were concentrated at room temperature under reduced pressure to afford a solid. For spectroscopic analysis, the products were washed with acetone and air dried. The products were characterized by FTIR and NMR.

The collected sulfonylurea solutions were concentrated under reduced pressure and redissolved in DCM and washed with water. The organic layer was concentrated under vacuum to afford a white solid. The products were purified with methanol and oven dried at 60° C. and characterized by FTIR and NMR.

Reaction 1: Flow Synthesis Preparation of Carbamate 2a from Amine 5a or Amine 5a HCl Salt

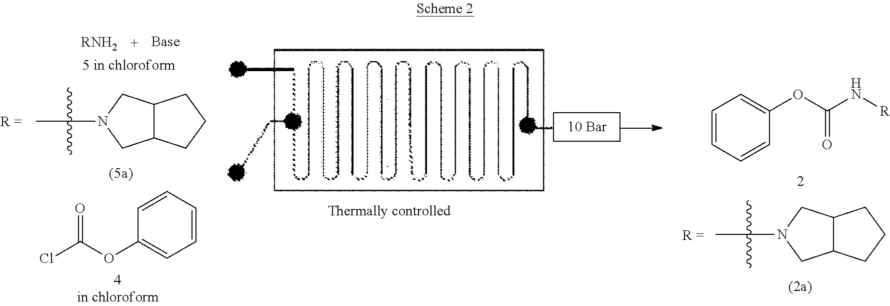

In one embodiment of the invention, as shown in Scheme 2 above, amine 5a was treated with chloroformate 4 in the presence of a base in a continuous flow system to afford the desired carbamate 2. The effect of residence time and temperature on the reaction was investigated in flow. The results of the investigations are presented in Table 1 below.

TABLE 1
The investigation of residence time and temperature
in the reaction towards carbamate.

| Entry | Res. Time (s) | Temp. (° C.) | Conversion (%)^{e} |
|---|---|---|---|
| 1 (a) | 15 | 0 | 78 |
| 2 (a) | 15 | r.t | 79 |
| 3 (a) | 30 | r.t | 100 |
| 4 (a) | 15 | 100 | 72 |
| 5 (a) | 60 | 100 | 59 |
| 6 (b) | 30 | r.t | 54 |
| 7 (d) | 30 | r.t | 78 |
| 8 (d) | 30 | 0 | 100 (96)^{f} |

Standard conditions:
(a) Amine 5a (0.1M, 1 Equiv.), DBU or TEA (1 Equiv.) and chloroformate 4 (1 Equiv.),
(b) Amine 5a (0.1M, 1 Equiv.), chloroformate 4 (1 Equiv.),
(d) Amine 5a (1M, 1 Equiv.), DBU or TEA (1 Equiv.), chloroformate 4,
^{e}Conversion determined by HPLC,
^{f}Number in parentheses corresponds to isolated yield.

Carbamate 2a was formed in 78% conversion at 0° C. in 15 s (Table 1, Entry 1). Increasing temperature to room temperature had little effect on the conversion (Table 1, Entry 2). However, an increase in temperature to 100° C. was accompanied by a decrease in conversion (Table 1, Entry 4 and 5). Further decrease in conversion was observed with increase in residence time (Table 1, Entry 5). This can be attributed to the thermal decomposition of carbamate 2a as confirmed by the formation of phenol. Performing the reaction at room temperature and 30 s residence time using 0.1 M solutions afforded full conversion (Table 1, Entry 3). However, the use of concentrated solutions (1 M) at room temperature was accompanied by a decrease in conversion (Table 1, Entry 7).

The carbamate formation reaction is exothermic and its exothermicity is more pronounced with increased reaction concentration. Since carbamate 2a is thermally unstable, it decomposes from the heat of reaction if the reaction temperature is not properly controlled. Consequently, carbamate 2a was prepared in full conversion and 96% isolated yield at 0° C. and 30 s using concentrated reagents (Table 1, Entry 8). Carbamate 2a was prepared in 54% yield in the absence of DBU (Table 1, Entry 6).

Amine 5a is commercially supplied as amine 5a HCl salt. With large scale synthesis carbamate 2a in mind, the inventors found it desirable to investigate the preparation of carbamate 2a directly from the amine 5a HCl salt without the need to first prepare the free amine 5a in a batch process. Amine HCl presents challenges with dissolution, in a particular in a micro flow system sensitive to system blockages, in particular with low temperature reaction conditions being preferred. It was therefore challenging to find an appropriate organic solvent to dissolve amine 5a HCl. Surprisingly, the inventors found that chloroform, dichloromethane and acetonitrile in the presence of equimolar base, including the organic base 1,8-Diazabicyclo[5.4.0]undec-7-ene (DBU), provided a satisfactory dissolution profile. In one example, amine 5a HCl salt was dissolved in chloroform in the presence of DBU pumped separately with chloroformate 4 into a 2 ml glass reactor to investigate the preparation of carbamate 2a (Scheme 2 and Table 2).

TABLE 2
The investigation of residence time and temperature in the
reaction towards carbamate 2a from amine 5a HCl salt

| Entry | Res. Time (s) | Temp. (° C.) | Conversion (%)^{d} |
|---|---|---|---|
| 1 (a) | 30 | 0 | 49 |
| 2 (a) | 60 | 0 | 52 |
| 3 (b) | 30 | 0 | 97(93)^{e} |
| 4 (b) | 60 | 0 | 100 |
| 5 (c) | 60 | 0 | 76 |

Standard conditions:
(a) Amine 5a HCl (1M, 1 Equiv.), DBU (1 Equiv.) and chloroformate 4 (1 Equiv.),
(b) Amine 5a HCl (1M, 1 Equiv.), DBU (2 Equiv.), chloroformate 4 (1 Equiv.),
(c) Amine 5a HCl (1M, 1 Equiv.), DBU (3 Equiv.), chloroformate 4 (1 Equiv.),
^{d}Conversion determined by HPLC,
^{e}Number in parentheses corresponds to isolated yield.

The preparation of carbamate 2a from amine 5a HCl in the presence of equimolar DBU was afforded in 49-52% conversion (Table 2, Entry 1 and 2). However, a conversion of 97% and 93% isolated yield was afforded in the presence of DBU (2 Equiv.) and full conversion was afforded in 60 s (Table 2, Entry 3 and 4). DBU (2 Equiv.) was found to be desirable to keep the free amine 5a in solution in chloroform as well as mopping the HCl formed in the reaction.

The use of DBU (3 Equiv.) resulted in a decrease in conversion (Table 2, Entry 5). The inventors managed to prepare carbamate 2a at a multigram scale in a Chemtrix Kiloflow® continuous flow system (24 ml total reactor volume) using the following reaction conditions Amine 5a HCl (1 Equiv.), DBU (2 Equiv.), chloroformate (1 Equiv.), 30 s residence time and 0° C. Carbamate 2a was afforded in 93% isolated yield with a throughput of 330 g/h.

Reaction 2: Flow Synthesis Preparation of Gliclazide 1a from Carbamate 2a

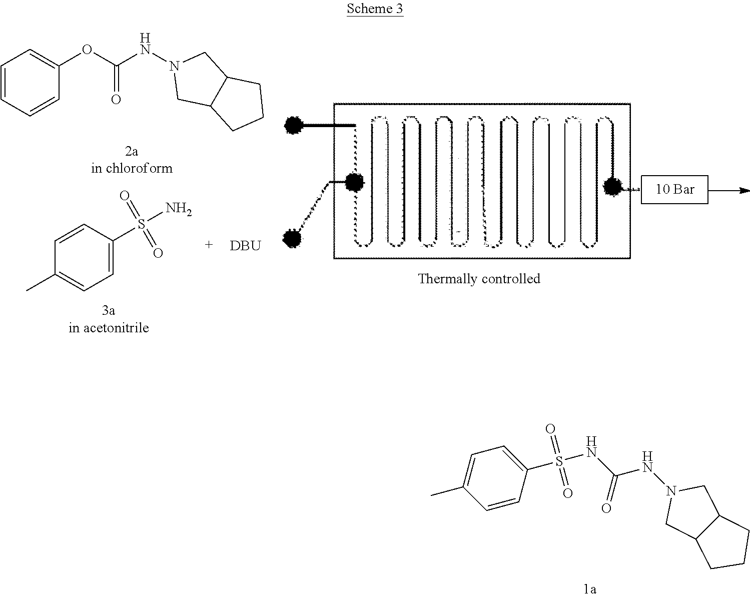 [SMILES: O=C(NN1CC2CCCC2C1)Oc1ccccc1 ; Cc1ccc(S(N)(=O)=O)cc1 ; "DBU" ; Cc1ccc(S(=O)(=O)NC(=O)NN2CC3CCCC3C2)cc1]

Following the successful preparation of carbamate 2a in flow, the inventors proceeded with the preparation of gliclazide 1a from carbamate 2a in flow. As is shown in Scheme 3 above, carbamate 2a was treated with sulfonamide 3a in the presence of a base in a 2 ml glass reactor. A summary of preliminary investigations is shown in Table 3.

TABLE 3
The investigation of residence time and temperature in
the reaction towards gliclazide 1a from carbamate 2a

| Entry | Res. Time (min) | Temp. (° C.) | Conversion (%)^{c} |
|---|---|---|---|
| 1 (a) | 1 | r.t | 8 |
| 2 (a) | 2 | r.t | 11 |
| 3 (a) | 2 | 80 | 46 |
| 4 (b) | 2 | 80 | 57 |
| 5 (b) | 2 | 100 | 49 |

Standard conditions:
(a) Sulfonamide 3a (0.5M, 1 Equiv.) premixed with DBU (2 Equiv.), carbamate 2a (1 Equiv.),
(b) Sulfonamide 3a (0.5M, 1 Equiv.) premixed with DBU (2 Equiv.), carbamate 2a (1.5 Equiv.),
^{c}Conversion determined by HPLC.

The treatment of carbamate 2a with sulfonamide 3a at room temperature and 1 min residence time afforded gliclazide 1a in a less than desirable conversion of 8% (Table 3, Entry 1). The conversion only increased slightly to 11% with an increase in residence time (Table 3, Entry 2). An increase in temperature to 80° C. resulted in increase in conversion to 46% (Table 3, Entry 3), which was still not considered sufficient. The inventors attempted to increase conversion by increasing the carbamate 2a equivalents, which improved gliclazide 1a synthesis (Table 3, Entry 4) to 57%. Further attempts by the inventors to improve the conversion towards gliclazide 1a included increasing the reaction temperature to 100° C. However, this change in reaction conditions was accompanied by a decrease in conversion (Table 3, Entry 5).

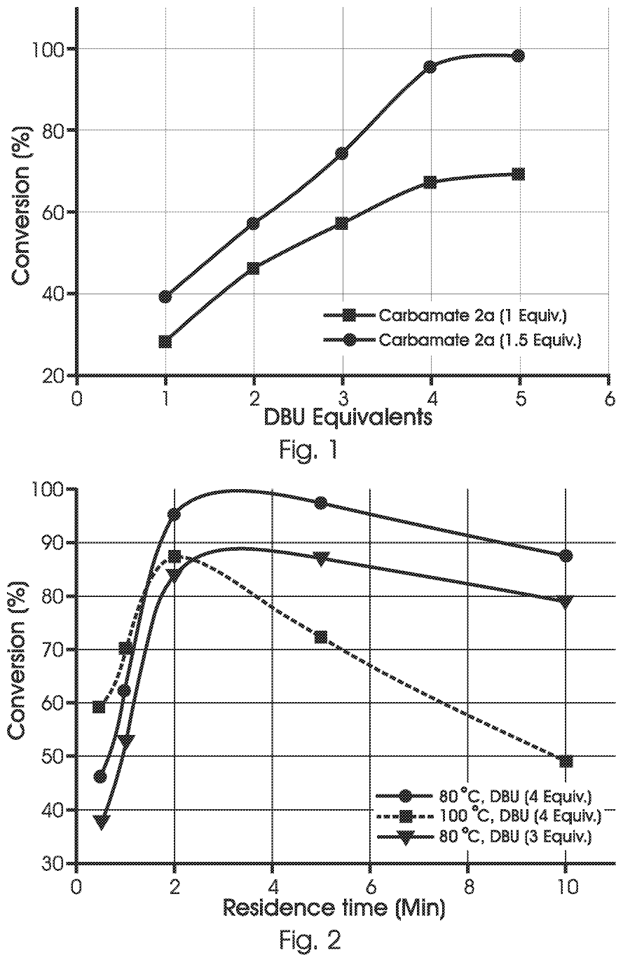

The inventors then surprisingly found that the correct molar equivalent ratio of DBU and carbamate 2a equivalents was central in obtaining the required conversion towards gliclazide 1a. FIG. 1 shows the results for investigations on the reaction when treating sulfonamide 3a (0.5 M) premixed with DBU with carbamate 2a at 80° C. in a 2 ml glass reactor for 2 min residence time.

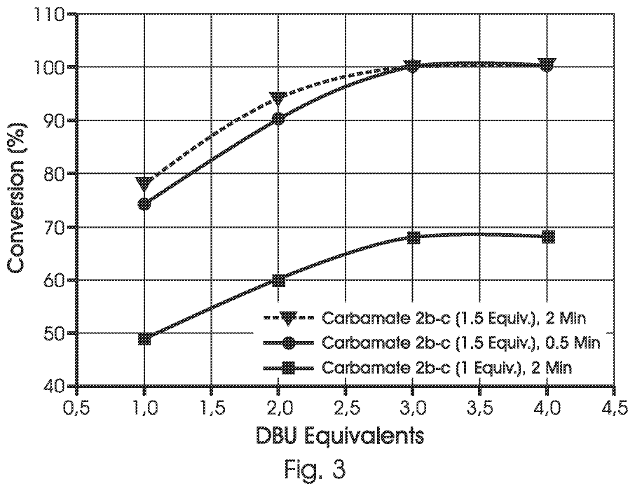

Gliclazide 1a formation improved significantly with an increase in both carbamate 2a and base (DBU) equivalents (see FIG. 1). A conversion of 98% was obtained in the presence of carbamate 2a (1.5 Equiv.) and DBU (5 Equiv.), while a conversion of 95% was obtained with carbamate 2a (1.5 Equiv.) and DBU (4 Equiv.). FIG. 2 shows the results obtained in the reactions of sulfonamide 3a (0.5 M, 1 Equiv.) with DBU (4 Equiv.) and carbamate 2a (1.5Equiv.), as well as sulfonamide 3a (0.5 M, 1 Equiv.) with DBU (3 Equiv.) and carbamate 2a (1.5 Equiv.) in a 2 ml glass reactor (FIGS. 3 and 2).

As can be seen from FIG. 2, conversion generally increased with increase in residence time, but only up to a certain point. The decrease in conversion with increase in residence time is more evident at 100° C. This can likely be attributed to the decomposition of carbamate 2a. In the particular flow synthesis setup used in this embodiment, the optimum conditions were found to be 80° C. and 2 min residence time using sulfonamide 3a (0.5 M, 1 Equiv.) premixed with DBU (4 Equiv.) and carbamate (1.5 Equiv.) to afford gliclazide 1a in and exceptional conversion of 95%. At the determined optimum conditions for gliclazide 1a, the reaction was scaled up in a Chemtrix Kiloflow® continuous flow system (18.4 ml total reactor volume) to afford gliclazide 1a in 93% conversion and 91% isolated yield with a throughput of 41 g/h.

Reaction 3: Continuous Multistep Flow Synthesis Method from Producing Gliclazide 1a From Amine 5a HCl

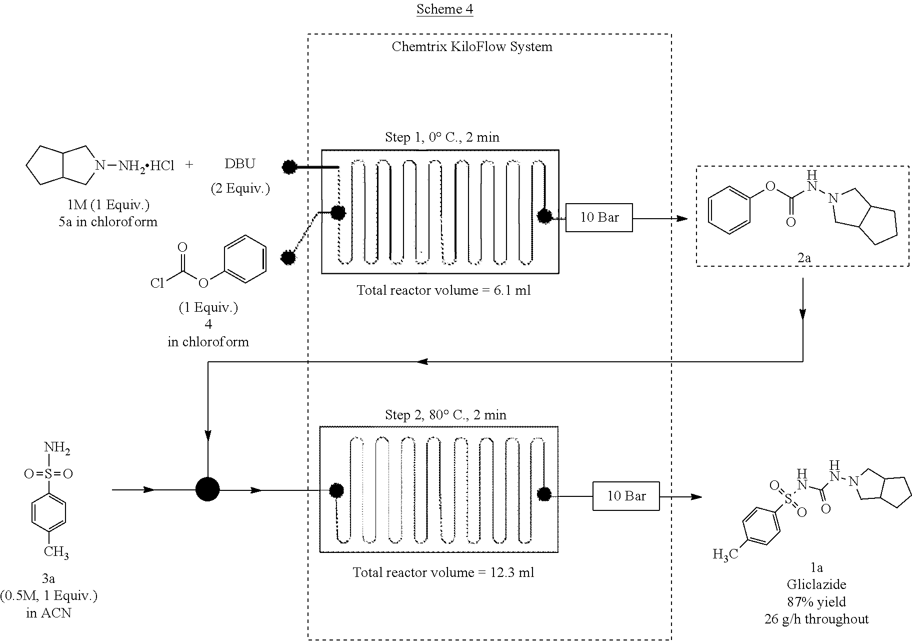

Based on the learnings from the experiments detailed above in respect of the single step reactions, the inventors successfully performed a multistep synthesis of gliclazide 1a from amine 5a HCl (1 M, 1 Equiv.) via the carbamate 2a intermediate. In this embodiment of a continuous multistep flow synthesis reaction, the process was performed in a Chemtrix Kiloflow® flow system (18.4ml total reactor volume) to afford gliclazide 1a in 90% conversion and 87% isolated yield with a throughput of 26 g/h (Scheme 4). It will be appreciated that the reagents and the reaction conditions shown in Scheme 4, including the use of amine 5a HCl salt, can be altered according to the teachings in the experiments above and other substitutions which the skilled person would consider to have a high likelihood of success.

### Reaction 4: Flow Synthesis Preparation of Carbamate 2b and Carbamate 2c From Amine 5b and 5c

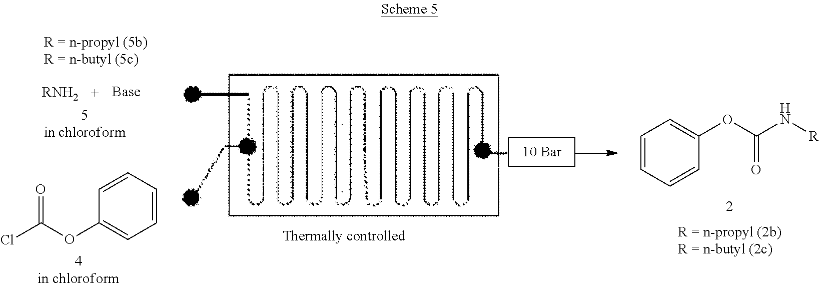

The respective carbamates 2b and 2c for chlorpropamide 1b and tolbutamide 1c were prepared starting from the conditions found to be favourable for the flow preparation of carbamate 2a (amine 5 0.1 M (1 Equiv.), DBU (2 Equiv.), 0° C. and 0.5 min) in a 2 ml reactor (Scheme 5 and Table 4). Due to close chemical reactivities of amines 5b and 5c, both carbamates 2b and 2c were formed in same conversions at comparable reaction conditions (Table 4).

TABLE 4
The preparation of carbamate 2 in flow

| | Chloroformate 4 | Amine 5 | | Carbamate 2 |
|---|---|---|---|---|
| Entry | Equivalents | Equivalents | Base | (%)^{b} |
| 1 | 1 | 1 | DBU (2 Equiv.) | 100 |
| 2 | 1 | 1 | DBU (1 Equiv.) | 100 |
| 3 | 1 | 1 | TBA | 100 |
| 4 | 1 | 1 | 0 | 55 |
| 5^{a} | 1 | 1 | TBA | 100 |

Standard conditions:
^{a}Amine 5b and 5c (2M, 1 Equiv.), Chloroformate 4 (1 Equiv.).
^{b}Conversion determined by HPLC.

The treatment of amine 5 (b and c) (0.1 M, 1 Equiv.) with phenyl chloroformate 4 in the presence of DBU (2 Equiv.) at 0° C. for 0.5 min afforded respective carbamates 2 (b and c) in 100% conversion (Table 4, Entry 1). Full conversion was still afforded when DBU (1 Equiv.) (Table 4, Entry 2). The use of an alternative base tributyl amine (TBA) (1 Equiv.) afforded carbamate 2 (b and c) in full conversion (Table 4, Entry 3). A conversion of 55% was obtained in the absence of a base as a result of the HCl formed that hinders the reaction by reacting with amine 5 (b and c) (Table 4, Entry 4). Carbamates 2 (b and c) were also prepared successfully using 2 M reagents in full conversion (Table 4, Entry 5). However, the use of concentrations above 2 M resulted in reactor clogging.

Carbamates 2b and 2c were prepared at multigram scale in a Chemtrix Kiloflow@ continuous flow system (24 ml total reactor volume) with amine 5 (b and c) (2M, 1 Equiv.), TBA (2 Equiv.), chloroformate 4 (1 Equiv.), 0.5 min residence time and 0° C. Both carbamate 2b and 2c were prepared at and isolated yield of 98% with a throughput of 516 g/h and 556 g/h respectively.

It will be appreciated by those skilled in the art that, in line with the preparation of carbamate 2a from amine 5a HCl salt, carbamates 2b and 2c could similarly be prepared in flow from the HCl salts of amine 5b and amine 5c.

Reaction 5: Flow Synthesis Preparation of Chlorpropamide 1b and tolbutamide 1c From Carbamate 2b and carbamate 2c

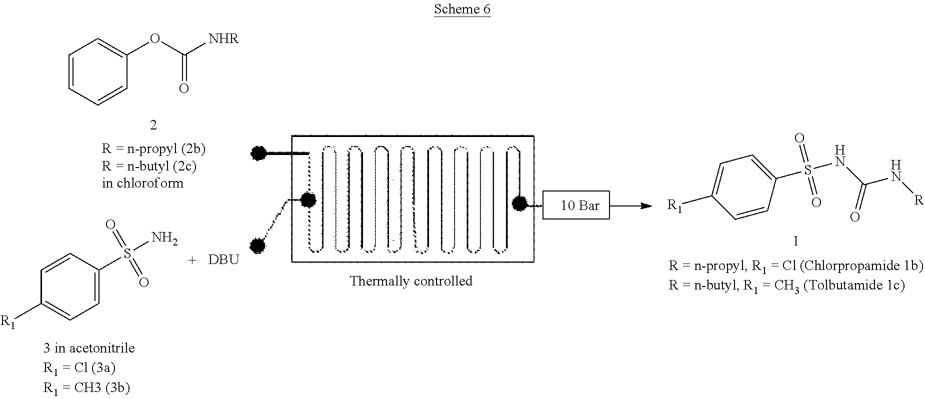

Chlorpropamide 1b and tolbutamide 1c were prepared from carbamates 2b and 2c respectively in a 2 ml glass reactor at the conditions found to be favourable for gliclazide 1a synthesis from carbamate 2a, i.e. (Sulfonamide 3a (1 M, 1 Equiv.) premixed with DBU (4 Equiv.), carbamate 2a (1.5 Equiv.), 80° C. and 2min residence time) (Scheme 6 and Table 5). Due to the close chemical reactivities of carbamates 2b and 2c and sulfonamides 3a and 3b, both chlorpropamide 1b and tolbutamide 1c were prepared in the same conversions at comparable reaction conditions (Table 5 and FIG. 3).

TABLE 5
The investigation of residence time and temperature
in the reaction towards chlorpropamide 1b and tolbutamide
1c from carbamate 2b and 2c^{a}

| Entry | Res. Time (min) | Temp. (° C.) | Conversion (%)^{d} |
|---|---|---|---|
| 1 | 2 | 80 | 100 |
| 2 | 2 | 50 | 76 |
| 3 | 5 | 50 | 81 |
| 4 (b) | 2 | 80 | 100 |
| 5 (c) | 2 | 80 | 100 |

Standard conditions:
^{a}Sulfonamide 3b and 3c (1M, 1 Equiv.) premixed with DBU (4 Equiv.), carbamate 2b and 2c (1.5 Equiv.),
(b) Sulfonamide 3a and 3b (1M, 1 Equiv.) premixed with DBU (3 Equiv.), carbamate 2b and 2c (1.5 Equiv.),
(c) Sulfonamide 3a and 3b (2M, 1 Equiv.) premixed with DBU (3 Equiv.), carbamate 2b and 2c (1.5 Equiv.),
^{d}Conversion determined by HPLC.

The treatment of sulfonamide 3b and 3c (1 M, 1 Equiv.) (4 Equiv.) with carbamate 2b and 2c (1.5 Equiv.), in the presence of DBU (4 Equiv.) at 80° C. and 2 min residence time afforded both chlorpropamide 1b and tolbutamide 1c in full conversion (Table 5, Entry 1). A decrease in temperature was accompanied by a decrease in conversion (Table 5, Entry 2). Increase in residence time slightly increased conversion (Table 5, Entry 3). Full conversion was still achieved with reduced DBU (3 Equiv.) (Table 5, Entry 4). Reaction concentration was successfully increased to 2M affording chlorpropamide 1b and tolbutamide 1c in full conversion (Table 5, Entry 5). These observations lead to more comprehensive investigations using 2 M reagents at 80° C. (see FIG. 3).

From FIG. 3 it can be seen that, conversion generally improved with increase in both DBU and carbamates 2b and 2c equivalents. The use of carbamates 2b and 2c (1 Equiv.) resulted in poor conversion. The optimum conditions, based on the flow synthesis setup utilised in this embodiment of the invention, were found to be sulfonamide (2 M, 1 Equiv.), DBU (3 Equiv.), carbamate 2b and 2c (1.5 Equiv.), 80° C. and 0.5 min residence time to afford both chlorpropamide 1b and tolbutamide 1c in full conversion.

The flow synthesis preparation of chlorpropamide 1b and tolbutamide 1c was scaled up in a Chemtrix Kiloflow® continuous flow system (18.4 ml total volume). A conversion of 98% and an isolated yield of 97% where achieved for both compounds, with a throughput of 592 g/h and 579 g/h for chlorpropamide 1b and tolbutamide 1c respectively.

### Reaction 6: Continuous Multistep Flow Synthesis Method From Producing Chlorpropamide 1b and Tolbutamide 1c From Amine 5b and 5c

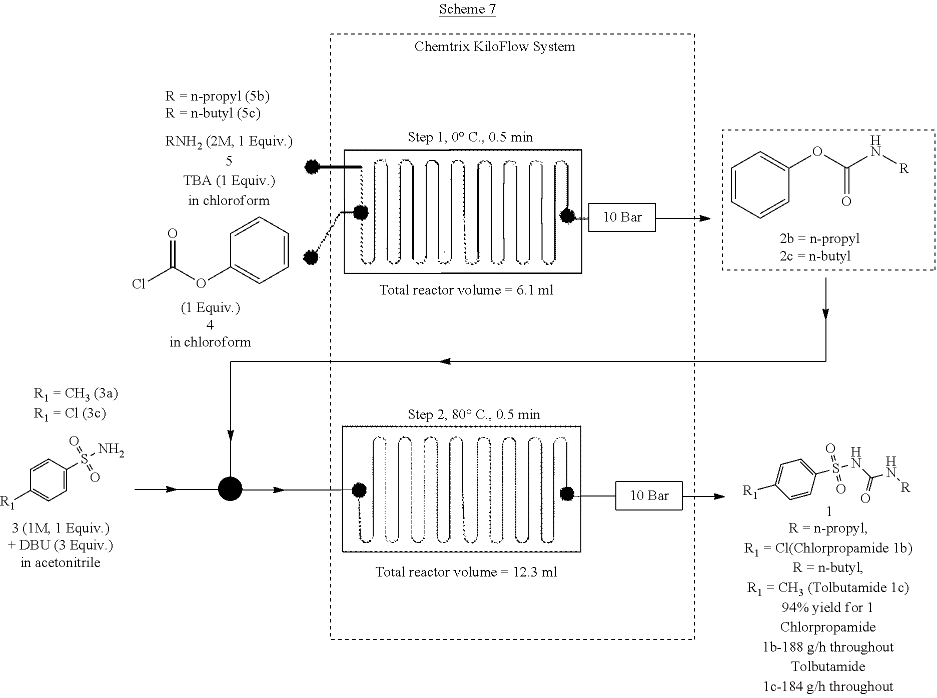

Based on the learnings from the experiments detailed above in respect of the single step reactions, the inventors successfully performed a multistep synthesis of chlorpropamide 1b and tolbutamide 1c from amine 5b and 5c via the carbamate 2b and 2c intermediates. This process was performed in a Chemtrix Kiloflow® flow system (18.4 ml total reactor volume) to afford chlorpropamide 1b and tolbutamide 1c in 95% conversion and 94% isolated yield with a throughput of 188 g/h and 184 g/h respectively (Scheme 7). It will be appreciated that the reagents and the reaction conditions shown in Scheme 7, including the use of free amines 5b and 5c, can be altered according to the teachings in the experiments above and other substitutions which the skilled person would consider to have a high likelihood of success.

The inventors have developed rapid and highly efficient flow synthesis methodologies for the preparation of the sulfonylurea compounds gliclazide, chlorpropamide and tolbutamide. The compounds were efficiently prepared throughputs of 82-592 g/h (91-97% yield) by single step synthesis. Furthermore, the compounds were efficiently prepared at multigram scale using continuous flow technology with a multistep synthesis at a throughput of 26-188 g/h and yields between 87-94% inside 4 minutes total residence time. In addition, it is particularly significant that the important carbamate intermediates for these compounds were prepared in very high throughputs of 330-556 g/h (93-98% yield).

### Spectroscopic Data

### Carbamate 2a

Following procedure 1, carbamate 2a was afforded as a white solid, mp 139-140° C.; ¹H NMR (400 MHz, CDCl₃) δ 7.26 (t, J=7.8 Hz, 2H), 7.15-6.98 (m, 3H), 5.93 (s, 1H), 3.14 (t, J=7.7 Hz, 2H), 2.54 (dd, J=8.3, 4.6 Hz, 2H), 2.41 (t, J=6.8 Hz, 2H), 1.57 (dt, J=15.3, 5.5 Hz, 3H), 1.51-1.30 (m, 3H). ¹³C NMR (101 MHz, CDCl₃) δ 150.8, 129.3, 125.2, 121.5, 115.4, 62.4, 40.3, 32.3, 25.5.

### Carbamate 2b

Following procedure 1, carbamate 2b was afforded as a white solid, mp 56-58° C.; ¹H NMR (400 MHz, CDCl₃) δ 7.27 (t, J=7.7 Hz, 2H), 7.11 (t, J=7.4 Hz, 1H), 7.05 (d, J =7.9 Hz, 2H), 5.01 (s, 1H), 3.15 (q, J=6.7 Hz, 2H), 1.51 (h, J=7.4 Hz, 2H), 0.88 (t, J=7.4 Hz, 3H). ¹³C NMR (101 MHz, CDCl₃) δ 153.6, 150.1, 128.2, 124.2, 120.6, 41.9, 22.0, 10.2.

Carbamate 2c

Following procedure 1, carbamate 2c was afforded as a white solid, mp 38-40° C.; ¹H NMR (400 MHz, DMSO-d6) δ 7.72 (t, J=5.7 Hz, 1H), 7.37 (t, J=7.7Hz, 2H), 7.19 (t, J=7.4 Hz, 1H), 7.09 (d, J=7.9 Hz, 2H), 3.07 (q, J=6.6 Hz, 2H), 1.46 (p, J=7.1 Hz, 2H), 1.33 (h, J=7.3 Hz, 2H), 0.90 (t, J=7.3 Hz, 3H). 13C NMR (101 MHz, DMSO-d6) δ 154.8, 151.6, 129.7, 125.3, 122.4, 31.8, 19.9, 14.1.

### Gliclazide 1a

Following procedure 1, gliclazide 1a was afforded as a white solid, mp 165-169° C.; ¹H NMR (400 MHz, CDCl₃) δ 8.70 (d, J=65.3 Hz, 1H), 7.88 (t, J=6.8 Hz, 2H), 7.24 (t, J=6.6 Hz, 2H), 6.23-5.77 (m, 1H), 3.22 (s, 1H), 2.75 (s, 1H), 2.62-2.39 (m, 3H), 2.35 (s, 3H), 1.87 (d, J=36.4 Hz, 2H), 1.37 (s, 5H). ¹³C NMR (101 MHz, CDCl₃) δ 151.1, 144.4, 135.3, 128.4, 127.3, 64.3, 39.3, 33.4, 29.9, 20.6.

### Chlorpropamide 1b

Following procedure 1, chlorpropamide 1b was afforded as a white solid, mp 128-130° C.; ¹H NMR (400 MHz, DMSO-d6) δ 10.66 (s, 1H), 7.91 (d, 2H), 7.69 (d, 2H), 6.52 (d, J=5.9 Hz, 1H), 2.91 (q, J =6.7 Hz, 2H), 1.34 (h, J=7.4 Hz, 2H), 0.76 (t, J=7.4 Hz, 3H). ¹³C NMR (101 MHz, DMSO) δ 151.8, 139.7, 138.5, 129.7, 129.6, 41.5, 22.9, 11.8.

### Tolbutamide 1c

Following procedure 1, tolbutamide 1c was afforded as a white solid, mp 128-130° C.; ¹H NMR (400 MHz, DMSO-d6) δ 10.45 (s, 1H), 7.78 (d, 2H), 7.41 (d, J=7.9 Hz, 2H), 6.42 (t, J=5.8 Hz, 1H), 2.94 (q, J=6.6 Hz, 2H), 2.39 (s, 3H), 1.30 (p, J=7.1 Hz, 2H), 1.17 (h, J=7.4 Hz, 2H), 0.82 (t, J=7.3 Hz, 3H). ¹³C NMR (101 MHz, DMSO) δ 151.7, 144.0, 137.9, 129.9, 127.7, 31.7, 21.5, 19.8, 14.0.

This above description of some of the illustrative embodiments of the invention is to indicate how the invention can be made and carried out. Those of ordinary skill in the art will know that various details may be modified thereby arriving at further embodiments, but that many of these embodiments will remain within the scope of the invention.

1. A flow synthesis process for producing a sulfonylurea compound of the Formula 1 or its pharmaceutically acceptable salts, 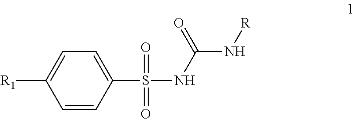
the process comprising the steps of:
a) preparing the carbamate of Formula 2 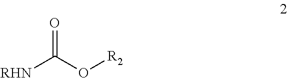
by reacting free amine RNH₂ or amine HCl salt with haloformate of Formula 4 in the presence of a base 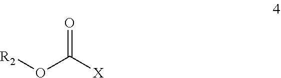
b) reacting the carbamate of Formula 2 with the sulfonamide of Formula 3 in the presence of a base 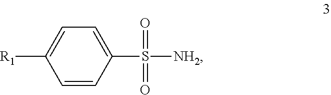
wherein
R₂ is selected from aryl and alkyl,
R₁ is selected from Cl and —CH₃,
R is selected from propyl, butyl, and 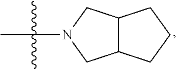 [SMILES: *N1CC2CCCC2C1]
and
X is selected from Cl, Br, and I.

2. The process according to claim 1, wherein the process is a continuous multi-step process without the isolation of any intermediates.

3. The process according to claim 1, wherein in step (a) the free amine RNH₂ or amine HCl salt to base molar ratio is about 1:1.5 to about 1:3.

4. The process according to claim 1, wherein the reaction of step (a) is performed using aryl chloroformate.

5. The process according to claim 4, wherein the reaction of step (a) is performed using phenyl chloroformate.

6. The process according to claim 1, wherein the reaction of step (a) is performed in an organic solvent selected from the group consisting of chloroform, dichloromethane, acetonitrile, and mixtures thereof.

7. The process according to claim 6, wherein the reaction of step (a) is performed in chloroform.

8. The process according to claim 1, wherein the base in step (a) and step (b) is independently selected from 1,8-Diazabicyclo[5.4.0]undec-7-ene (DBU), trimethylamine (TEA), and tributyl amine (TBA).

9. The process according to claim 8, wherein the base used in step (a) and step (b) is the same.

10. The process according to claim 1, wherein the reaction of step (a) is thermally controlled to a temperature equal to about room temperature or less.

11. The process according to claim 10, wherein the reaction of step (a) is thermally controlled to a temperature between about 0° C. and about room temperature.

12. The process according to claim 1, wherein the free amine RNH₂ or amine HCl salt has a concentration of about 0.1M to about 2M.

13. The process according to claim 1, wherein the sulfonamide has a concentration of about 0.1M to about 2M.

14. The process according to claim 1, wherein in step (b) the sulfonamide to carbamate molar ratio is about 1:1 to about 1:2.

15. The process according to claim 14, wherein in step (b) the sulfonamide to carbamate molar ratio is about 1:1.5.

16. The process according to claim 1, wherein in step (b) the sulfonamide to base molar ratio is about 1:2 to about 1:5.

17. The process according to claim 1, wherein the reaction of step (b) is thermally controlled to a temperature between about 70° C. and about 100° C.

18. The process according to claim 17, wherein the reaction of step (b) is thermally controlled to a temperature of about 80° C.
